(19) United States
(12) Patent Application Publication
(10) Pub. No.: US 2003/0203839 A1
(43) Pub. Date: Oct. 30, 2003

(54) Immune enhancing composition containing lactoferrin

(76) Inventors: Marian L. Kruzel, Houston, TX (US); Gilbert A. Castro, Houston, TX (US)

Correspondence Address: Kurt S. Myers, 7634 Braesdale, Houston, TX, 77071, US

(21) Appl. No.: 10/456,457

(22) Filed: Jun. 6, 2003

Related U.S. Application Data
(63) Continuation-in-part of application No. 09/430,484, filed on Oct. 29, 1999, now Pat. No. 6,613,741.

(51) Int. Cl. A61K 38/40; A61K 31/70

(52) U.S. Cl. 514/6; 514/23

(57) ABSTRACT

The composition of the present invention provides a novel use of the iron binding protein lactoferrin as a medicament to prevent the progression of systemic inflammatory response syndrome (SIRS) into sepsis, severe sepsis, septic shock and multiple organ failure in mammals. More particularly, the present invention is the use of lactoferrin for the manufacture of a medicament for the treatment of the metabolic imbalance in ICU patient.

### RELATED APPLICATIONS

This application is a continuation-in-part application of Ser. No. 09/430,484, filed Oct. 29, 1999—which is based on PCT application PCT/US98/09053, filed Apr. 30, 1998, entitled “Method for Treating Aseptic SIRS in Humans and other Animals”, which in turn is based on provisional application Ser. No. 60/045,521 filed May 3, 1997 entitled “Use of Lactoferrin for Prophylaxis and Therapy of the Systemic Inflammatory Response System in Animals and Humans”, both of which are incorporated herein by reference. This application also relates to U.S. Pat. No. 6,066,469, based on Ser. No. 08/724,586, filed Sep. 30, 1996, entitled “Cloning, Expression and Uses of Human Lactoferrin”, which in turn is a continuation of U.S. Ser. No. 08/238,445, filed May 5, 1994, which in turn is a CIP of U.S. Ser. No. 08/132,218, filed Oct. 6, 1993, which in turn is a continuation of U.S. Ser. No. 07/489,186, filed Mar. 8, 1990, all of which are incorporated herein by reference.

### FIELD OF THE INVENTION

The present invention relates generally to the iron binding protein lactoferrin administered either systemically or orally to prevent the progression of systemic inflammations into sepsis in humans and animals. More specifically, it relates to lactoferrin as an adjunctive medicament (formula) aimed to increase the medical benefits of desired therapy in human and non-human animals.

### BACKGROUND OF THE INVENTION

The immune system plays in mammals a central role in protection against microbial infections, neoplasia, as well as many age-related disorders. The mucosal surface of the gastrointestinal tract is one of the first important interfaces between the pathogens or various dietary antigens and the host. Thus, the intestine is the site of intense immunologic activities and the challenge is to maintain a disease-free state in the face of chronic antigen exposure. Normal immune homeostasis depends on coordinated interactions among the various lymphoid, phagocytic and somatic cells which comprise the immune system. In general, these interactions are tightly regulated to obtain a balance between the need to eliminate harmful insults and the need to avoid damaging autoimmune response. The maintenance of homeostasis is essential for cellular integrity and depends on the ability of our body to induce proper inflammatory responses. The ultimate goal of inflammation is to dispose of both the initial cause of injury and its consequence. In the intestine of a normal healthy individual however, inflammation has its own “physiologically justified” chronic character. Again, this is a reflection of continuous exposure of epithelium to myriads of dietary antigens and pathogens. In fact, this constitutes oral tolerance which is defined as the immunologic mechanism by which the mucosal immune system maintains unresponsiveness to many antigens. Homeostasis is a state of equilibrium in the internal environment. The integrity of such system is continuously disturbed by stimuli that tend to create an internal imbalance. In response to prolonged stimuli, the compensatory mechanisms often do not restore the balance. This may, consequently lead to the activation of self-perpetuating, autodestructive mechanisms promoting systemic inflammation and its sequels, including death. Regardless the insult cause the internal environment responds to those insults by activating thermoregulatory mechanisms that coincide with the production and release of many immunomodulatory substances. Cytokines, prostaglandins, and different growth factors and hormones are released from specific cells to restore the internal metabolic balance. In the normal controlled inflammatory response mediators are released timely in conjunction with specific inhibitors to contain the response. If these responses are activated in an uncontrolled fashion with dissemination via the circulation, over the period of time, organs distant from the initial insult can be affected to produce multiple organ failure.

The systemic inflammatory response syndrome (SIRS) has been recently defined by Rangel-Frausto et al. in a paper entitled “The natural history of the Systemic Inflammatory Response Syndrome (SIRS); A prospective study” published in JAMA (1995), 273:117-123. Accordingly, patient experiencing two of the four symptoms listed below is qualified as a SIRS patient;

1. Temperature>38° C. or <36° C.

2. Heart rate>90 beats/min.

3. Respiratory rate>20 breaths/min.

4. White blood cell count>12.0×108 L, <4.0×108 L, or >0.10 immature forms (bands).

SIRS may develop into sepsis when bacteria are confirmed in circulation.

The significance of lactoferrin in animals health and disease has been the subject of several reviews. A most recent publication entitled “Lactoferrin and immunologic dissonance: Clinical implications” has been published by Kruzel M L and Zimecki M. in Arch Immunol Ther Exp 2002;50:325-333. Lactoferrin is a multifunctional protein expressed by mammals in a variety of cell types under different mechanisms of control. The primary function of lactoferrin seems to be a protection against pathogenic bacteria. By virtue of sequestering iron, lactoferrin may control development of potential infections. In addition, it can kill a wide variety of Gram-negative and Gram-positive bacteria by direct interaction with the cell surface, a mode of action that is not dependent on iron. Lactoferrin is thought to be an important component of the defense system, active at mucosal surfaces, including the gastrointestinal tract. Various immunoregulatory and anti-infective roles for lactoferrin have been reviewed by J. Brock in an article entitled “Lactoferrin: a multifunctional immunoregulatory protein?” and published in Immunology Today (1995), 16:417-419 and by B. Lonnerdal and S. lyer in an article entitled “Lactoferrin: Molecular Structure and Biological Function” and published in Ann. Rev. Nutr., (1995)15:93-110.

Although, considerable data from in vitro experiments indicate several physiological roles for lactoferrin, there is less evidence concerning its actual physiological function from in vivo studies. For example, in a review by Roy D. Byens and Werner R. Bezwoda entitled “Lactoferrin and the inflammatory response” and published in the book: Lactoferrin: Structure and Function, pp 133-141, (1994), a relationship between plasma lactoferrin and granulocyte activity in sepsis is mentioned. However, the biological function of the significant amounts of lactoferrin in plasma of septic patients is as yet incompletely understood. In another review entitled “The role of lactoferrin as an anti-inflammatory molecule” by Bradley E. Britigan, Jonathan S. Serody, and Myron S. Cohen and published in the book: Lactoferrin: Structure and Function, pp 143-156, (1994), the role of lactoferrin in inflammation is suggested to be played at two different levels: (i) as an antioxidant, capable of binding free iron, and (ii) as an endotoxin scavenger, capable of reducing lipopolysaccharide (LPS)-induced toxicity. Furthermore, the ability of lactoferrin to bind LPS in vitro has been confirmed by E. Elass-Rochard, A. Roseanu, D. Legrand, M Trif, V. Salmon, C. Motas, J. Montreuil and G. Spik in an article entitled “Lactoferrin-lipopolysaccharide interaction: involvement of the 28-34 loop region of human lactoferrin in the high-affinity binding to Escheria coli 055B5 lipopolysaccharide”, published in Biochem. J. (1995)312:839-845. However, in vivo studies have to confirm lactoferrin's role in those internal metabolic responses during inflammatory processes.

In another article entitled: “Lactoferrin can protect mice against a lethal dose of Escherichia coli in experimental infection in vivo” by T. Zagulski, P. Lipinski, A. Zagulska, S. Broniek and Z. Jarzabek, published in 1989 in Br. J. Exp. Path., 79:697-704, the use of lactoferrin is disclosed to increase the survival of mice injected with a lethal dose of bacteria. However there is no disclosure that lactoferrin given orally or intravenously has any effect on the gut function and structure to give such protection.

A high level of lactoferrin in plasma has been suggested to be a predictive indicator of sepsis-related, morbidity and mortality (reviewed by Bayens et al., 1994). Increased neutrophil count is paralleled by increased in plasma lactoferrin, and visa versa (Bayens et al., 1986). This correlation is not surprising, because lactoferrin is a constituent of secondary granules in neutrophils and can be released from these cells by exogenous stimili such as bacteria.

Relevant patents are also silent as to the role of lactoferrin in insult-induced metabolic activity.

U.S. Pat. No. 4,977,137 of Nichols et al. discloses milk lactoferrin as a dietary ingredient which promotes growth of the gastrointestinal tract of human infants and newborn nonhuman animals immediately on birth. Nichols discusses the use of lactoferrin in the management of short gut syndrome, an anatomical dysfunction rather than an insult-induced metabolic imbalance.

U.S. Pat. No. 5,240,909 of Nitsche relates to the use of lactoferrin as an agent for the prophylactic and therapeutic treatment of the toxic effects of endotoxins. Nitche discloses that the lactoferrin used according to his invention has the ability to neutralize endotoxin and must have bound to it either iron or another metal to be effective.

U.S. Pat. No. 5,066,491 of Stott et al. encompasses a method of disease treatment utilizing a therapeutically effective product produced from ordinary milk whey.

Also, there are various patents regarding use of lactoferrin, or its natural source such as colostrum, in numerous dietary formulas.

U.S. Pat. No. 4,342,747 by Liotet et al. relates to the therapeutic use of colostrum for the external ophthalmic use.

U.S. Pat. No. 5,750,149 by Gobbi relates to pharmaceutical and/or dermocosmetic compositions containing equine colostrum.

U.S. Pat. Nos. 6,258,383; 6,410,058; 6,475,511 by Gohlke et al. relate to use of lactoferrin as a composition with colostrum and modified citrus pectin or beta glucan in a form of chewable tablets or lozenges.

None of these applications disclose the use of lactoferrin to prevent a progression of systemic inflammations into septic conditions.

### SUMMARY OF THE INVENTION

The present invention provides a novel composition of immune enhancing medicament containing the iron binding protein lactoferrin to prevent the progression of the systemic inflammatory responses into sepsis in humans and other mammals. Lactoferrin, as an immune enhancing medicament, also considered as an adjuvant, is administered orally (by mouth, feeding tube or spray) or systemically (intravenously) for human patients in a critical care situations (e.g. Intensive Care Units—ICU).

### DESCRIPTION OF THE DRAWINGS

FIG. 1 illustrates histological sections of mouse jejunum stained with hematoxylin and eosin to visualize intestinal structures during experimental endotoxemia a) non-infected, saline control, b) non-infected, lactoferrin-treated, c) infected, saline-treated, d) infected, lactoferrin-treated.

### DESCRIPTION OF THE PREFERRED EMBODIMENTS

Inflammation is fundamentally a protective response to cell injury. When excessive in magnitude or duration, however, the otherwise beneficial effects of inflammation may be deleterious, impacting negatively on the recovery or healing of the host. Neutrophils are the first phagocytic cells to arrive at the site of injury to ingest bacteria, dead cells, and cellular debris. When tissue damage occurs, particularly if it is induced by infection during trauma, vascular effects are immediate. The tissue becomes inflamed at the site of injury, with the tissue spaces and the lymphatics blocked by fibrin clots. The fluid barely flows through the inflamed tissue, therefore the spread of bacteria and/or their toxic products is delayed. Unlike the immune responses, which may take days to develop, the vascular effects of inflammation occur in seconds and coincide with the burst of inflammatory cytokines, such as TNF-α and IL-1β from activated monocytes/macrophages. Subsequent to the release of cytokines is an acute increase in neutrophils in the blood. Within an hour after inflammation begins, the bone marrow may increase the production of fresh neutrophils up to fivefold. Large numbers of neutrophils begin to invade the tissues that attract these cells. The feedback control of inflammation begins with the degranulation of neutrophils and a release of lactoferrin, which in turn inhibits the production and release of TNF-α, IL-1β, NO and granulocyte/macrophage colony stimulating factor (GM-CSF). However, when the insult or tissue damage is severe, the natural feedback control mechanism of inflammation is insuffient and the patient while exhibiting SIRS may progress into sepsis, multiple organ failure or death.

According to the present invention, the feedback control mechanism of inflammation is enhanced by the administration of lactoferrin as a primary medicament. By blocking the deleterious effects of the pro-inflammatory cytokines, lactoferrin provides a feedback mechanism for the metabolic imbalance during development of systemic inflammation, and thus, can be used in accordance with the present invention in a method for treating or preventing such insult-induced metabolic imbalance that otherwise may proceed to various disorders, including sepsis. Also, such a treatment can be given in conjunction with other treatments to increase the medical benefits (adjunctive therapy). Thus lactoferrin treatment, according to the present invention, is considered as both adjunctive and adjuvant therapy, acting via innate immune system signaling to active desired innate and adaptive immune responses. Lactoferrin used according to the present invention stimulates both short and long term protective responses against broad categories of environmental insults. The purpose of both adjunctive and adjuvant treatment is to assist the primary treatment and to increase the chances of a cure (examples of adjuvant therapy: chemotherapy, radiation or hormone replacement).

The gastrointestinal tract, and to some degree the respiratory system (lungs), may be viewed as an ecological system that acts to maintain balance be involved in maintaining this balance. The first is a non-specific structural barrier provided by the epithelial layer of the gastrointestinal or bronchial mucosae. The second component involves functional immunological elements found in the mucosal and submucosal compartments. When the integrity of these organs is disrupted by invasive pathogens or by trauma, a myriad of pro-inflammatory mediators is released from the mucosal sites that exert actions in the tissue. The organ responses to such insults are immediate to protect the internal environment from enteric bacteria crossing the gut barrier and inducing systemic responses. Therefore, it is a great importance to preserve the structure and function of these organs during initial stages of such metabolic imbalance to avoid the systemic translocation of the enteric or bronchial bacteria.

According to the present invention both the gastrointestinal tract and respiratory system are considered the key target organs to provide compensatory mechanisms to any type of insult-induced metabolic imbalance. The present invention, by in vivo experiments on gut, shows that lactoferrin attenuates the severity of the insult-induced metabolic imbalance, thereby protecting the development of severe hypo- or hyperactivity that often leads to chronic inflammation or systemic inflammatory response syndrome. Furthermore, it has been found that lactoferrin can be used in accordance with the present invention in a method for protecting intestinal functions during insult-induced metabolic imbalance. The evidence presented herein confirms that lactoferrin administered either enterally or parenterally helps to maintain physiological balance in normal and pathologic situation.

Insult is defined herein as any intervention in the internal environment including microbial, viral or parasitic infections; stress; trauma; chemotherapy; irradiation; insufficient or excessive nutrient intake; invasive or non-invasive medical procedures, any of which cause metabolic imbalance, a state of disturbed integrity of the internal system. Usually, such metabolic imbalance is referred to as hypo- or hyper-activity of the internal system.

Lactoferrin is one of the most abundant proteins found at mucosal surfaces and within secondary granules of neutrophiles in all vertebrates. The highest concentration of lactoferrin has been found in mammary glands of lactating females. The sequence homology between human and other species lactoferrins is 50% to 70%. Bovine milk lactoferrin, which is commercially available, is about 69% identical to its human counterpart. Due to this fact, the clinical application of bovine lactoferrin in humans is limited to oral administration; any type of systemic administration of bovine lactoferrin in humans would cause highly antigenic reaction.

Lactoferrin for use in a present invention may be human lactoferrin from human breast milk or extracted from milk of other animals such as bovine lactoferrin from cow's milk or whey. Due to severe limitations on availability of large quantities of human breast milk and the FDA requirements, it may be difficult to develop a commercial production of clinically acceptable natural human lactoferrin. Consequently, recombinant DNA technology is considered the best solution to obtaining large quantities of reliable human or bovine lactoferrins which would be consistent in production, uniform in its biological properties, and non-pathogenic. Of particular interest for systemic human applications would be a human lactoferrin produced in an expression system providing human type glycosylation, such as described by Choi B K, Bobrowicz P, Davidson R C, Hamilton S R, Kung D H, Li H, Miele R G, Nett J H, Wildt S, Gerngross T U. Use of combinatorial genetic libraries to humanize N-linked glycosylation in the yeast Pichia pastoris. Proc Natl Acad Sci U S A. 2003;100:5022-5027.

The preferred lactoferrin is lactoferrin expressed in a yeast expression system such as Pichia pastoris or Hansenula polymorpha, or in a eukaryotic expression system. The preferred lactoferrin is described in U.S. Pat. No. 6,066,469, entitled “Cloning, Expression and Uses of Human Lactoferrin” and its two divisional applications U.S. Pat. No. 6,277,817 B1 and 6,455,687 B1, both entitled “Human lactoferrin”. Other recombinant lactoferrins are described in U.S. Pat. Nos. 5,571,691; 5,571,697; and 5,571,896, all of which are incorporated herein by reference. Lactoferrin produced by any of these expression system is further purified and reconstituted into its natural physiologic formula, such as saline solution.

Lactoferrin is administered in accordance with the present invention either systemically (intravenously, intramuscularly) or orally (by mouth, through feeding tube or in spray), as a medicament. Preferable medicaments or formulations of the present invention comprise lactoferrin in a pharmaceutical or nutritional carrier such as, water, physiologically compatible phosphate buffer—saline (PBS), glucose, glycols, lipids or other non-denaturing solutions capable of sustaining tertiary structure of lactoferrin. The lactoferrin is preferably present in the formulation at a level of 0.01 milligram to 2 milligram, more preferably between 0.1 to 1 milligram, based on 1 milliliter or 1 gram of the carrier. An effective amount of lactoferrin varies depending on the individual treated, severity of the metabolic imbalance and the form of administration. Preferable in treating individual human patients, a single or twice daily dose of 0.01 milligram to 20 milligrams, more preferable 0.1 milligram to 1 milligram of lactoferrin per kilogram of body weight is administrated.

According to the present invention, lactoferrin can be incorporated in formulation with any drug therapy (adjunctive protocol) and delivered simultaneously with other multipurpose formulas. It is postulated that functional foods provide a variety of health benefits including anti-microbial, anti-inflammatory, anti-carcinogenic, hypocholesterolemic or hypertension controlling effects. It has been a long term challenge to identify what ingredient constitutes specific activity, what is the optimal concentration, and possibly, whether any composition with other ingredients can increase the performance of such formula. The combination of lactoferrin with functional foods provides better health benefit. Indeed, the nutritional immunomodulation in disease and health promotion is one of the most important factors in hospital's recovery rooms today. The interrelationships among functional foods, immunity, disease and health are very complex consisting of issues such as oral tolerance and the effects of specific nutrients on immune function or immune profiles in different physiologic conditions.

The most preferably, lactoferrin is administered in admixture with one of the commercially available formulas, generally referred as Enteral Nutrition Formulas used in the ICU. According to the present invention, lactoferrin can be used as an improvement in the following formulas: Accupep HPF, Advera, Alitraq, Amin-Aid, Attain, Carnation Instant Breakfast, Carnation Instant Breakfast No Sugar Added, Casec, CitriSource, Citrotein, Compleat Modified, Compleat Regular, Comply, Criticare HN, Crucial, Deliver 2.0, DiabetiSource, Elementra, Ensure, Ensure with Fiber, Ensure High Protein, Ensure HN, Ensure Plus, Ensure Plus HN, Entrition Half-Strength, Entrition HN, Fiberlan, Fibersource, Fibersource HN, Glucema, Glytrol, Great Shake, Great Shake Jr, Hepatic-Aid II, Immun-Aid, Impact, Impact with Fiber, Introlan, Introlite, Isocal, Isocal NH, Isolan, Isosource, Isosource HN, IsoSource VHN, Isotein HN, Jevity, Kindercal, Lipisorb, Magnacal, MCT Oil, Menu Magic Instant Breakfast, Menu Magic Milk Shake, Meritene, Microlipid, Moducal, Nepro, NuBasics, NuBasics with Fiber, NuBasics Plus, NuBasics VHP, Nutren 1.0, Nutren 1.5, Nutren 2.0, Nutren 1.0 with Fiber, NutriHep, Nutrilan, NutriVent, Osmolite, Osmolite HN, Pediasure, Pediasure with Fiber, Peptamen, Peptamen Junior, Peptamen VHP, Perative, Polycose, Pre-Attain, ProBalance, Profiber, ProMod, Promote, Promote with Fiber, Propac Plus, Protain XL, Pulmocare, Reabilan, Reabilan HN, Replete, Replete with Fiber, Resource, Resource Plus, Respalor, SandoSource Peptide, 206 Shake, Sumacal, Suplena, Sustacal, Sustacal Basic, Sustacal with Fiber, Sustacal Plus, Sustagen, Tasty Shake, Toerex, TraumaCal, Traum-Aid HBC, Travasorb HN, Travasorb Renal Diet, Travasorb STD, TwoCal HN, Ultracal, Ultralan, Vital High Nitrogen, Vitaneed, Vivonex Pediatric, Vivonex Plus, Vivonex T.E.N., or other enteral nutritional formulas used as nutritional replacements. All these formulas can be taken by mouth or through a feeding tube to increase a chance of cure.

The effectiveness of lactoferrin in the treatment or prevention of insult-induced metabolic imbalance according to the present invention is demonstrated below in LPS-induced mouse model. Injection with LPS, a derivative of the cell wall of Gram negative bacteria, is commonly used as the insult for study of SIRS and sepsis. The following objectives were evaluated: (i) safety of a long term oral administration of lactoferrin and (ii) the effects of lactoferrin on the metabolic activity during LPS-induced endotoxemia, all in a mouse model.

The procedures and methods for determining the physiological function of the gut under different type of insults are summarized as follows. These procedures have been developed to measure the state of the gut as normal, returned to normality, or pathologic using saline-treated animals as a control. Human lactoferrin has been used replaceable with its bovine counterpart. Only male CF-1 mice (Harlan, Houston, Tex.), are used throughout this investigation. Mice are housed in groups of three per cage and are given stock diet (F6 Rodent Diet 8664, Teklad, Madison, Wis.) and water at libitum.

### EXAMPLE 1

Naive mice were gavaged daily with human lactoferrin (1 mg/100 μl saline) for 21 days each morning. Their control counterparts were given 100 μl of saline for the same time period. After 21 days of treatment all mice were killed and jejunal segments were obtained for electrophysiological measurements and histological examination.

Food consumption and cumulative weight gain in mice fed lactoferrin were similar in both lactoferrin and saline-treated mice. Basal electrophysiological parameters of mouse jejunum [intestinal transepithelial resistance (R), potential difference (PD) and short circuit current (SCC)] were not altered by long term administration of lactoferrin. Likewise, intestinal glucose absorption, and Cl⁻ secretion induced by 5-HT (serotonin), CCh, or histamine were not affected by three weeks administration of lactoferrin. Also, the histology of the intestine was not altered in mice fed lactoferrin.

### EXAMPLE 2

The effect of human lactoferrin on the development of LPS-induced endotoxemia in mice was determined by examining survival of mice upon challenge with a lethal dose of LPS. Naive mice were injected once intraperitoneally with 150 μl of saline solution lactoferrin (7.5 mg/mouse) one hour before or after LPS challenge. This illustrates both a prophylactic and therapeutic treatment. Their control counterparts were given 150 μl of saline. Bacterial LPS (E. coli, Serotype 0111:B4) was given intraperitoneally at the lethal dose of 1.5×10⁶ endotoxic units per mouse. The survival of mice was monitored over the period of time of four weeks (n=6 per each group).

| Treatment | Survival (%) |
|---|---|
| LPS | 16.6 |
| Lactoferrin followed by LPS (prophylactic application) | 83.3 |
| LPS followed by Lactoferrin (therapeutic application) | 66.6 |

A single intraperitoneal dose of lactoferrin (7.5 mg) administered 1 hour before or after LPS challenge significantly increased the survival of mice.

### EXAMPLE 3

The effectiveness of oral administration of lactoferrin on survival of mice subjected to lethal injection of LPS was determined in the following experiment. Naive mice were gavaged with 150 μl of saline solution of bovine lactoferrin (7.5 mg/dose) for three days prior or after LPS challenge. Their control counterparts were given 150 μl of saline. Bacterial LPS (E. coli, Serotype 0111:B4) was given intravenously at the lethal dose of 1.5×10⁶ endotoxic units per mouse. The survival of mice was monitored over the period of time of four weeks (n=6 per each group).

| Treatment | Survival (%) |
|---|---|
| LPS | 37.5 |
| Lactoferrin followed by LPS (prophylactic application) | 50 |
| LPS followed by Lactoferrin (therapeutic application) | 83.3 |

Oral administration of lactoferrin for three days prior or after intravenous administration of LPS increased significantly the survival of mice.

### EXAMPLE 4

The effect of lactoferrin on electrophysiological parameters of gut function in mice challenged with a lethal dose of LPS was determined in the following experiment. Naive mice (n=6 per each group) were injected intraperitoneally with a lethal dose of LPS (1.5×10⁶ endotoxic units per mouse). One hundred microliters (100 μl) of saline solution of human lactoferrin (10 mg/ml) was given to mice enterally by gavaging at 1 hour, 3 hours and 6 hours post-LPS challenge. Their counterparts were given saline. Twenty four hours later the blood samples were collected, mice were sacrificed and jejunal segments removed for electrophysiological and histological measurements.

Treatment of mice with lactoferrin had no effect on the electrophysiological characteristics of jejunal epithelium. Resistance (R) of the intestinal tissue following LPS challenge was significantly lower in both lactoferrin treated mice and saline control. Mice fed lactoferrin and challenged with LPS showed a moderate increase in short circuit current when compared with saline control. PD was comparable for all groups. Glucose absorption was about 30% higher for LPS-challenged animals with no significant difference between lactoferrin treated and saline control. Cl— secretory response to 5-HT and CCh was also elevated in both LPS groups.

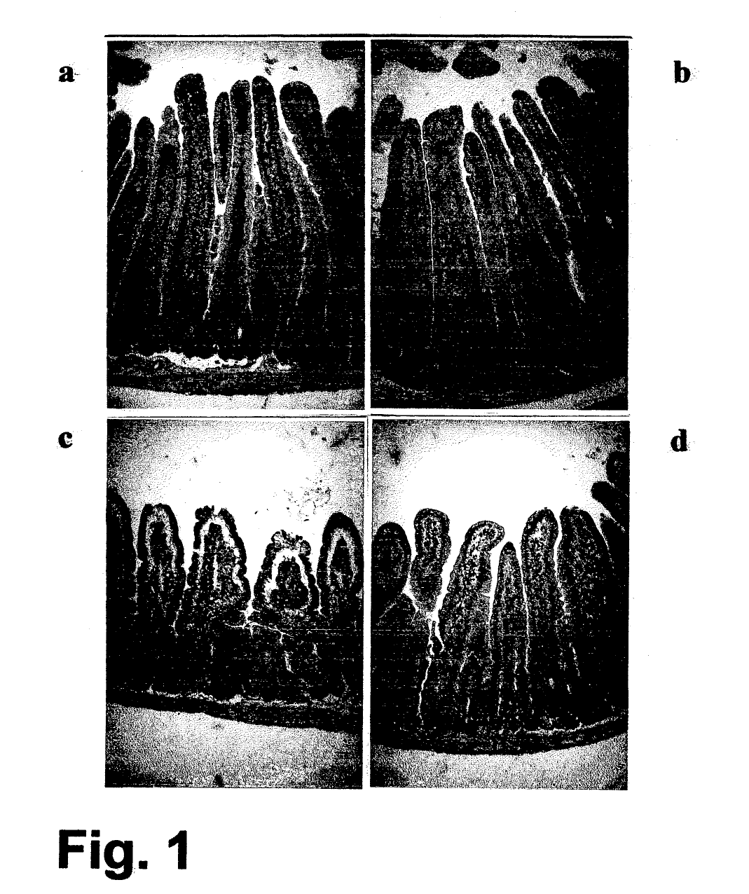

The intestinal epithelium of mice injected with LPS exhibited severe vacuolar degeneration in control animals with shortening and shrinking of the villi and expansion of the crypts. There were heavy inflammatory infiltrates in the tunica mesenteris of control animals (FIG. 1; a—saline control; b—LF control; c—LPS; d—LPS/LF). In the lactoferrin-treated mice vacuolar degeneration was less pronounced with the epithelium resembling the highly polarized, resorbtive epithelium of non-infected mice.

This protective effect of lactoferrin on gut function and structure during experimental endotoxemia correlates with significant reduction of cytokines in plasma. It has been shown that lactoferrin attenuates the release of TNFα and IL-1β into plasma by more than 50% when measured 2 hours post LPS challenge. However, only an insignificant decrease in the concentration of those cytokines was observed in the intestinal tissue homogenates.

### EXAMPLE 5

Five hundred grams (500 g) of commercially available highly purified (greater than 90%) bovine milk lactoferrin (e.g. product of Morinaga, DMV International Nutritionals, or Tatua Co-Operative Dairy Company Ltd), or any commercially available human recombinant lactoferrin (e.g. product of FerroDynamics, Inc.) is placed into a commercial mixer and one hundred litters (100 L) of phosphate buffered saline (PBS) solution, such as Dulbecco's PBS without calcium and magnesium chloride (Gibco/Invitrogen Corporation; Cat. No. 14-190-144) is added. The solution is stirred in room temperature until all visible particles are dissolved and disappeared, then it is passed through 0.2 micron filter, such as Nalgen product, or equivalent, for the final product sterilization. The solution, which is an equivalent of 0.5% lactoferrin and referred hereinafter as LFS is packed into a sterile plastic bags, disposable pouches, spray containers or bottles, all under the sterile conditions. The procedure is performed with precautions to the powders and dust that are formed. The same procedure as above may be used to make solutions which have an equivalent of 0.01%-1.0% lactoferrin.

LFS as per Example 5 is given to subjects intravenously, by mouth, through feeding tube or by oral, nasal or alveolar spray.

### EXAMPLE 6

Five hundred grams (500 g) of commercially available highly purified (greater than 90%) bovine milk lactoferrin (e.g. product of Morinaga, DMV International Nutritionals, or Tatua Co-Operative Dairy Company Ltd), or any commercially available human recombinant lactoferrin (e.g. product of FerroDynamics, Inc.) is placed into a commercial mixer and one hundred liters (100 L) of 10% Travasol solution manufactured by Baxter Healthcare Corporation, containing essential and nonessential amino acids, is added (Each 100 ml of 10% Travasol contains: Leucine 730 mg, Isoleucine 600 mg, Lysine 580 mg, Valine 580 mg, Phenylalanine 560 mg, Histidine 480 mg, Threonine 420 mg, Methionine 400 mg, Tryptophan 180 mg, Alanine 2070 mg, Arginine 1030 mg, Glycine 1030 mg, Proline 680 mg, Serine 500 mg, Tyrosine 40 mg). The solution is stirred in room temperature until all visible particles are dissolved and disappeared, then it is passed through 0.2 micron filter, such as Nalgen product, or equivalent, for the final product sterilization. The solution, which is an equivalent of 0.5% lactoferrin and referred hereinafter as LFTravasol is packed into a sterile plastic bags, disposable pouches, spray containers or bottles, all under the sterile conditions. The procedure is performed with precautions to the powders and dust that are formed.

The same procedure as above may be used to make solutions which have an equivalent of 0.01%—1.0% lactoferrin. Also, the same procedure as above may be used to substitute Travasol with other physiologic solutions for intravenous administration (e.g. dextrose or glucose).

### EXAMPLE 7

Five hundred grams (500 g) of commercially available and highly purified (greater than 90%) bovine milk lactoferrin (e.g. product of Morinaga, DMV International Nutritionals, or Tatua Co-Operative Dairy Company Ltd), or any commercially available and highly purified human recombinant lactoferrin (e.g. product of FerroDynamics, Inc.) is placed into a commercial mixer and one hundred liters of any commercially available enteral feeding formula (e.g. TraumaCal—product of mead Johnson) as an improvement to the formula. The solution is mixed for 30 minutes at room temperature and aliquated into plastic bags or bottles under the steril conditions. The procedure is performed with precautions to the powders and dust that are formed. The same procedure as above may be used to make solutions which have an equivalent of 0.01%-1.0% lactoferrin.

TraumaCal (product of Mead Johnson Nutritionals), comprising the following: Water, corn syrup, calcium caseinate, soybean oil, sodium caseinate, sugar, medium chain triglycerides (MCT oil), soy lecithin, potassium citrate, magnesium chloride, sodium chloride, artificial flavor, potassium chloride, calcium carbonate, carrageenan, vitamin A palmitate, vitamin D3, vitamin E acetate, sodium ascorbate, folic acid, thiamin hydrochloride, riboflavin, niacinamide, vitamin B6 hydrochloride, vitamin B12, biotin, calcium pantothenate, vitamin K1, choline bitartrate, potassium iodide, ferrous sulfate, cupric sulfate, zinc sulfate, manganese sulfate.

It is evident from all the foregoing examples that lactoferrin given either orally or systemically is capable of modulating the gut function during insult-induced metabolic imbalance by protecting intestinal epithelium. We have demonstrated that such protection of gut function have inhibitory systemic effect on development of autodestructive mechanisms including death. Both prophylactic and therapeutic applications of lactoferrin have been shown to be effective in accordance with the present invention.

It is understood that the examples and embodiments described herein are for illustrative purposes only and that various modifications or changes in light thereof will be suggested to persons skilled in the art and are to be included within the spirit and purview of this application and scope of the appended claims.

1. A composition for the prophylactic treatment of the progression of systemic inflammatory response syndrome (SIRS) into sepsis, severe sepsis, septic shock and multiple organ failure in mammals comprising a solution containing a therapeutically effective amount of lactoferrin.

2. A composition according to claim 1 wherein said solution is saline.

3. A composition according to claim 1 wherein said solution is 10% Travasol.

4. A composition according to claim 1 wherein said solution is enteral nutrition formula.

5. A composition according to claim 1 wherein said administration is enteral.

6. A composition according to claim 1 wherein said administration is parenteral.

7. A composition according to claim 1 wherein said lactoferrin is human lactoferrin.

8. A composition according to claim 1 wherein said lactoferrin is bovine lactoferrin.

9. Use of lactoferrin for the manufacture of a medicament for the treatment or prevention of insult-induced metabolic imbalance in a mammal.

10. The use according to claim 9, wherein said mammal is a human.

11. A composition which comprises a solution of lactoferrin in glucose wherein said lactoferrin is present in a concentration of 0.01%-1% lactoferrin.

12. A composition for an adjunctive therapy which comprises a solution containing a therapeutically effective amount of lactoferrin.

13. A composition according to claim 12 wherein said an adjunctive therapy is an Enteral Nutritional Formula.
